(19) United States
(12) Patent Application Publication
(10) Pub. No.: US 2008/0058605 A1
(43) Pub. Date: Mar. 6, 2008

(54) TUNNEL VISION GYNOCOLOGICAL EXAMINATION DEVICE

(76) Inventor: Bradford Tyler Sorensen, Manhattan Beach, CA (US)

Correspondence Address: BRADFORD T. SORENSEN, 515 13TH STREET, MANHATTAN BEACH, CA, 90266, US

(21) Appl. No.: 11/469,103

(22) Filed: Aug. 31, 2006

(51) Int. Cl. A61B 1/32

(52) U.S. Cl. 600/208

(57) ABSTRACT

The present invention is a more effective and functional device and method for opening of the vagina for examination and treatment of the cervix and viewing the vaginal lining during a gynecological exam, which gently and safely opens the vagina, forming an expanding clear tunnel, which enlarges to display a 360 degree view of the vaginal lining and provides a mirror to offer an additional angle of view of the cervix for improved gynecological diagnosis and treatment.

### BACKGROUND AND SUMMARY OF THE INVENTION

The present invention is a device that creates a clear expanded tunnel which exposes the cervix and interior lining of the vagina for improved gynecological examinations.

Specifically, the current invention provides a device that consists of a spiral roll of flexible, durable, clear, sheet material that is inserted into the vagina and unrolled by a drive mechanism to form a clear cylindrical tunnel which opens up to expose and give easy access to the cervix at the end of the tunnel and displays the lining of the vagina through the clear walls of the tunnel to the gynecologist during an exam. The current invention allows 360 degree viewing of the interior of the vagina for improved diagnosis and direct, open, cylindrical, access to the cervix for treatments. As the clear cylindrical tunnel expands within the vagina, the interior diameter grows slightly larger than the outer diameter which naturally holds the truncated conical shaped tunnel inside the pinching muscle at the opening of the vagina. The drive mechanism has the multi-functions of providing controlled opening and dosing of the clear tunnel, providing a ratchet mechanism that locks the tunnel at any desired diameter, provides a vacuum fluid removal suction function and provides an automatic enlargement and reduction of the tunnel maintaining a slightly larger interior diameter than exterior diameter truncated conical shape for conical tunnel retention in the vagina. The device incorporates a rounded insertion tip that has a mirror surface positioned within the tip to reveal a reflection of the cervix to give the gynecologist an additional cervix viewing angle for improved diagnosis and treatment.

Historically gynecology exams involved the use of duck billed speculums which are inserted into the vagina and pried open by means of a hand lever or screw levers. The opaque metal split open upper and lower duck bill portions tend to hide the lining of the vagina and have a tendency to spread the lining vertically forming narrow vertical slot with stretched vertical walls of exposed vaginal lining on the sides of the duckbill blades and the cervix exposed at the deep tips of the duckbill blades. These speculums have had many variations; like the additions of multiple duckbill blades, lights, fiber-optics, light tubes and cameras over the years, but the basic duckbilled vaginal speculum has not changed since the 1800s. The current invention provides complete instant 360 degree visual access for accurate gynecological diagnosis and cervical treatments.

Historically the vaginal speculum was a metal device that had to be cleaned and sterilized between each use. A speculum having threaded adjustment screws and hinge connectors that create small confined areas where germs can be trapped can lead to an infection or contamination risk. The current invention is manufactured and packaged in a sterile environment, opened, used once and disposed of, to eliminate the opportunity for infection or contamination.

Historically, duckbill speculum devices have placed pressure points on the vagina as the blades pry open the vaginal lining in two directions. The current invention creates an expanding clear tunnel that spreads out the pressure over a 360 degree contact thereby reducing the pressure in pounds per square inch for increased comfort.

This device can be adapted to all mammals.

This device can be adapted to examine all bodily orifices.

This device can be adapted to become a retractor to hold tissue open during an operation.

### PREVIOUS ART

In the past, various patents have issued relating to Speculum devices. In particular, U.S. Pat. No. 6,719,687, issued on Apr. 13, 2004, to Van Der Weegen teaches a vaginal speculum with a seal that blows up the birth canal like a balloon and has a dome shaped seal that allows the gynecologist to turn the vaginal opening into a sealed ball and socket joint to examine the interior of the vagina and the cervix in a pressurized manner. While this inflated vagina concept is very innovative, this device does not allow viewing of the vaginal lining from the midpoint to the vaginal opening because the balloon shaped interior sphere arches away from the view of the gynecologist. A pressurized, balloon shaped vagina does not allow access of a surgeon's tools or hands to perform treatments.

The present invention displays 100% of the vaginal lining and cervix and opens a non-pressurized tunnel access to the cervix for PAP smear samples and treatments and operations to the cervix.

U.S. Pat. No. 6,712,761, issued on Mar. 30, 2004, to Borodulin teaches a standard duckbilled speculum with a lens mounted inside the opening.

The present invention provides a wide open tunnel in which to place lenses or cameras to document physical conditions.

U.S. Pat. No. 6,669,654, issued on Dec. 30, 2003, to Diokno teaches a standard duckbilled speculum with removable and slidable blades with measurement markings for measuring vaginal prolapses.

The present invention visual tunnel is printed or embossed with measurement markings for scale reference and has color samples for color matching reference for more accurate diagnosis. The present invention is furnished with measurement markings for measuring vaginal prolapses.

U.S. Pat. No. 6,432,049, issued on Aug. 13, 2002, to Banta teaches a standard duckbilled speculum with a light mounted inside the opening.

The present invention has a built in light source that floods the entire structure of the visual tunnel with light that is conducted through the clear material that makes up its architecture. The entire present invention lights up to illuminate for better diagnosis and treatment.

U.S. Pat. No. 6,432,048, issued on Aug. 13, 2002, to Francois teaches a standard duckbilled speculum with lateral wall retractors mounted inside the opening.

The present invention improves on lateral wall retractors by providing a 360 degree support of the vaginal wall lining for instant diagnosis.

U.S. Pat. No. 6,416,467, issued on Jul. 9, 2002, to McMillin teaches a standard duckbilled speculum with 3 blades instead of 2.

The present invention replaces blades with a flexible clear tunnel.

U.S. Pat. No. 6,048,308, issued on Apr. 11, 2000, to Strong teaches a standard duckbilled speculum with 3 blades instead of 2.

The present invention eliminates the pressure points of blades with a single cylindrical tunnel that spreads out the force onto a very large surface area thereby reducing the pounds per square inch of pressure for increased comfort U.S. Pat. No. 6,004,265, issued on Dec. 21, 1999, to Hsu teaches a standard duckbilled speculum with a light guide.

The present invention utilizes every part of its structure as a light guide.

U.S. Pat. No. 5,873,820, issued on Feb. 23, 1999, to Norell teaches a standard duckbilled speculum curved to lock inside the vaginal pinch muscle.

The present invention visual tunnel opens from a cylindrical to a conical shape with the larger end of the truncated cone inside the vaginal pinch muscle for automatic device retention until the cone is collapsed to the smaller diameter cylinder for removal.

U.S. Pat. No. 5,465,709, issued on Nov. 14, 1995, to Dickie teaches a standard duckbilled speculum with a chemical light stick illumination device.

The present invention could also utilize a chemical light stick light source to provide illumination to the light guide structure without the use of batteries or a light emitting diode.

U.S. Pat. No. 5,458,595, issued on Oct. 17, 1995, to Tadir teaches a standard duckbilled speculum with a laser.

The present invention would be a perfect combination for a laser because the wide open tunnel exposes the cervix for laser therapies and treatments. Laser light heats up pigmentation and colors. Therefore, the clear material of the present invention would be unaffected by laser light wavelengths.

U.S. Pat. No. 5,179,937, issued on Jan. 19, 1993, to Lee teaches a standard duckbilled speculum that is disposable.

The present invention is disposable to eliminate the chance of infection.

U.S. Pat. No. 5,052,372, issued on Oct. 1, 1991, to Lee teaches a standard duckbilled speculum that has a single control wheel.

The present invention also has a single control wheel or knob with an automatic indexing locking and release lever

U.S. Pat. No. 4,971,036, issued on Nov. 20, 1990, to Collins teaches a standard duckbilled speculum that is heated and vibrates for muscle relaxation and comfort while examining the vagina.

The present invention can also be heated, vibrating, pH sensing, pelvic pressure sensing and carry an electric diode for deadening pain.

U.S. Pat. No. 5,377,667, issued on Jan. 3, 1995, to Patton teaches a pivoted arm speculum with 6 blades that spread and contract.

The present invention expands with surface area as the diameter gets larger thereby compensating for the increase in surface pressure as the vaginal lining stretches tighter and tighter.

U.S. Pat. No. 2,083,573, issued on Jun. 15, 1937, to Morgan teaches a pivoted arm speculum with 4 blades that spread and contract.

The present invention makes a round opening: instead of a slot, a triangle, a square, a pentagram or a hexagram shaped opening.

U.S. Pat. No. 380,745, issued on Apr. 10, 1888, to Chamberlin teaches a pivoted arm speculum with 4 blades that spread and contract.

The present invention has no blades that look like weapons or stabbing sticks.

U.S. Pat. No. 167,666, issued on Sep. 14, 1875, to Higbee teaches a standard duckbilled speculum.

The present invention inserts at a small diameter and grows larger in diameter instead of going in at a large scale and stretching with a concentrated spreading force.

U.S. Pat. No. 55,511, issued on Jun. 12, 1866, to Leutz teaches a standard duckbilled speculum with 4 blades instead of 2.

The present invention comes packaged in sterile lubrication for easy insertion, expansion, contraction removal and disposal. Previous art speculums could be infected by the lubrication process.

The present invention provides innovative improvements over these examples of previous art and other previous art by introducing a more effective and functional device and method for opening and examining the interior of the vagina or other bodily cavity, which gently and safely opens the vaginal opening and vaginal lining, forming an expanding tunnel, which enlarges in diameter for the 360 degree view of the interior of the body cavity with a wide open access to the cervix or the interior of the body cavity.

The present invention also provides a mirror to offer an additional angle of view of the cervix for improved gynecological diagnosis and treatment.

### OBJECTS OF THE INVENTION

The first object of the present invention is to provide an improved alternative to the use of the duckbill speculum circa 1875.

The second object of the present invention is to provide a device which gently and safely opens the vaginal opening and vaginal lining, forming an expanding tunnel, which enlarges in diameter for the 360 degree view of the interior of the body cavity with a wide open access to the cervix or the interior of the body cavity.

The third object of the present invention is to provide a built in light source that floods the entire structure of the visual tunnel with light that is conducted through the clear material that makes up its architecture.

The fourth object of the present invention is to provide utilization of every part of its structure as a light guide.

The fifth object of the present invention is to provide an orifice viewing device with a chemical light stick light source to provide illumination to the light guide structure without the use of batteries or a light emitting diode.

The sixth object of the present invention is to provide a wide open tunnel in which to place lenses or cameras to document physical conditions.

The seventh object of the present invention is to provide a device that is a perfect combination for a laser because the wide open tunnel exposes the cervix for laser therapies and treatments. Laser light heats up pigmentation and colors. Therefore, the clear material of the present invention would be unaffected by laser light wavelengths. Laser treatments could be made through the walls of the clear tunnel.

The eighth object of the present invention is to provide measurement markings for measuring vaginal prolapses.

The ninth object of the present invention is to provide a visual tunnel that is printed or embossed with measurement markings for scale reference and color samples for color matching reference for more accurate diagnosis.

The tenth object of the present invention is to provide a visual tunnel that opens from a cylindrical to a truncated conical shape with the larger end of the truncated cone inside the vaginal pinch muscle for automatic device retention until the cone is collapsed to the smaller diameter cylinder for removal.

The eleventh object of the present invention is to provide a device with a single control wheel or knob with a spring loaded automatic indexing locking and release lever.

The twelfth object of the present invention is to provide a device that can be heated, vibrating, pH sensing, pelvic pressure sensing and carry an electric diode for deadening pain.

The thirteenth object of the present invention is to provide a device that has no blades that look like weapons or stabbing sticks.

The fourteenth object of the present invention is to replace blades with a flexible clear tunnel.

The fifteenth object of the present invention is to eliminate the pressure points of blades with a single cylindrical tunnel that spreads out the force onto a very large surface area thereby reducing the pounds per square inch of pressure for increased comfort.

The sixteenth object of the present invention is to provide a device that expands with surface area as the diameter gets larger thereby compensating for the increase in surface pressure as the vaginal lining stretches tighter and tighter.

The seventeenth object of the present invention is to provide a device that inserts at a small diameter and grows larger in diameter instead of going in at a large scale and stretching with a concentrated spreading force.

The eighteenth object of the present invention is to provide a device that makes a round opening; instead of a slot, a triangle, a square, a pentagram or a hexagram shaped opening.

The nineteenth object of the present invention is to provide improvements on previous art lateral wall retractors by providing a 360 degree support of the vaginal wall lining for instant diagnosis.

The twentieth object of the present invention is to provide a device that replaces current speculums that have opaque blades with a clear tunnel that does not hide the vaginal lining from the view of the gynecologist during an exam.

The twenty first object of the present invention is to provide a device with a mirror to offer an additional angle of view of the cervix for improved gynecological diagnosis and treatment.

The twenty second object of the present invention is to provide a device that is disposable to eliminate the chance of infection.

The twenty third object of the present invention is to provide a device that comes packaged in sterile lubrication for easy insertion, expansion, contraction removal and disposal. Previous art speculums could be infected by the lubrication process.

### BRIEF DESCRIPTION OF THE DRAWINGS

FIG. 1 is a perspective view of an embodiment of the present invention tunnel vision gynecological examination device inside the lining of the vagina in a rolled configuration (solid lines) and an expanded configuration (dotted lines).

FIG. 2 shows a side view of an embodiment of the present invention tunnel vision gynecological examination device inside the lining of the vagina in a rolled configuration (solid lines) and an expanded configuration (dotted lines).

FIG. 3 shows a perspective end view of an embodiment of the present invention tunnel vision gynecological examination device as seen from the viewpoint of a gynecologist doing a vaginal examination.

### PREFERRED EMBODIMENT COMPONENT LIST

10 tunnel vision speculum
11 vaginal opening
12 vaginal lining
13 rounded tip
14 orifice opener
15 mirror
16 cervix
17 reflection of cervix
18 spreader flange
19 spiral rolled clear tunnel
20 handle
21 finger knob
22 ratchet teeth
23 ratchet release
24 gear drive shaft
25 drive shaft sleeve
26 front drive gear teeth
27 rear drive gear teeth
28 front drive gear holes
29 rear drive gear holes
30 alignment guide slot
31 rolled tunnel
32 unrolled tunnel
33 rolled forward diameter
34 unrolled forward diameter
35 rolled rear diameter
36 unrolled rear diameter
37 forward minimum diameter locking hole
38 rear minimum diameter locking hole
39 vaginal pinching muscle
40 vacuum
41 vacuum tube
42 vacuum manifold drain
43 vacuum manifold intake slots
44 vacuum manifold
45 soft rounded conformal edge
46 light source
47 light source power connector

### DETAILED DESCRIPTION OF THE EXEMPLARY EMBODIMENT(S)

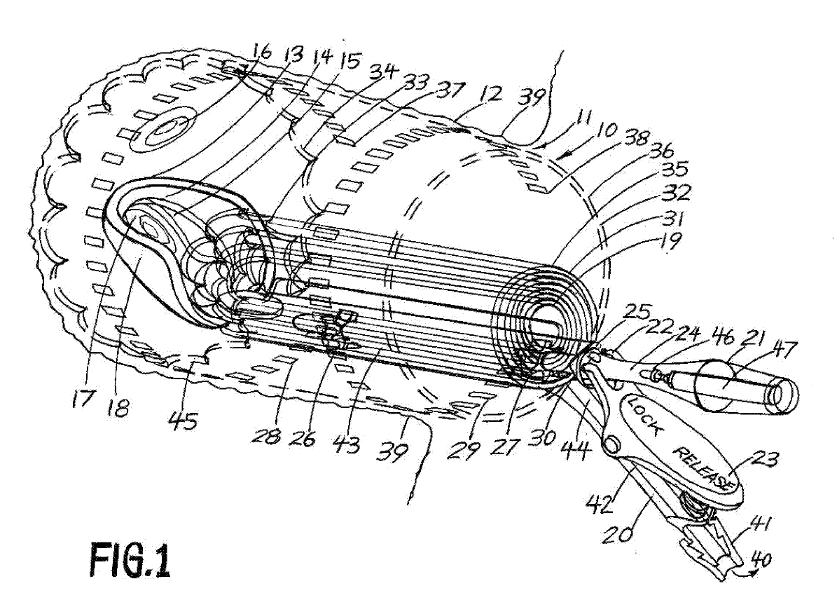

FIG. 1 shows a perspective view of an embodiment of the present invention tunnel vision gynecological examination device inside the lining of the vagina in a rolled configuration (solid lines) and an expanded configuration (dotted lines) illustrating the 10 tunnel vision speculum inserted into the 11 vaginal opening and surrounded by the 12 vaginal lining with the 13 rounded tip of the 14 orifice opener positioned deep inside the 12 vaginal lining where a 15 mirror mounted to the inside of the 14 orifice opener is located in a perfect position and angle below the 16 cervix to provide a 17 reflection of cervix to the gynecologist viewing through the 18 spreader flange and the 19 spiral rolled clear tunnel displaying a complete 360 degree view of the 12 vaginal lining and the 16 cervix and the 17 reflection of the cervix at once. The gynecologist controls the movement of the 10 tunnel vision speculum by grasping the 20 handle. In order to gain a wider view of the 12 vaginal lining, the gynecologist rotates the 21 finger knob, after disengaging the 22 ratchet teeth by depressing the 23 ratchet release on the 20 handle, which causes the rotation of the 24 gear drive shaft within the 25 drive shaft sleeve causing the 26 front drive gear teeth and the 27 rear drive gear teeth to engage and move the 28 front drive gear holes and the 29 rear drive gear holes within the 30 alignment guide slot causing the 31 rolled tunnel to move in a manner that increases the diameter of the spiral resulting in an 32 unrolled tunnel where the 33 rolled forward diameter is enlarged to form the 34 unrolled forward diameter and the 35 rolled rear diameter is enlarged to form the 36 unrolled rear diameter causing the tunnel to expand in the 12 vaginal lining to give the gynecologist an excellent view of the vaginal orifice for examinations, treatments, operations and tests. The tunnel vision speculum is provided with a 37 forward minimum diameter locking hole and a 38 rear minimum diameter locking hole in order to limit the minimum diameter the 19 spiral rolled clear tunnel may be reduced in size to avoid taking the spiral wound plastic past the point where the elastic memory of the plastic flex capability is exceeded and the plastic will no longer spring out into a tunnel shape again. The 34 unrolled forward diameter is larger than the 36 unrolled rear diameter causing a conical shape to be formed inside the 12 vaginal lining where the 39 vaginal pinching muscle has a tendency to hold the device inside the 11 vaginal opening until the 32 unrolled tunnel is reduced in size to form the 31 rolled tunnel which is small enough in diameter to be removed. The 10 tunnel vision speculum may be provided with a 40 vacuum source in order to remove fluids through a 41 vacuum tube that is connected to a 42 vacuum manifold drain that suctions fluids that collect in 43 vacuum manifold intake slots in the 44 vacuum manifold. The 10 tunnel vision speculum is provided with a flexible 45 soft rounded conformal edge to safely and comfortably expand within the 12 vaginal lining to afford the greatest access with the minimum discomfort. The 10 tunnel vision speculum may be equipped with a 46 light source and 47 light source power connector for the purpose of illuminating the interior of the 12 vaginal lining for improved ocular acuity.

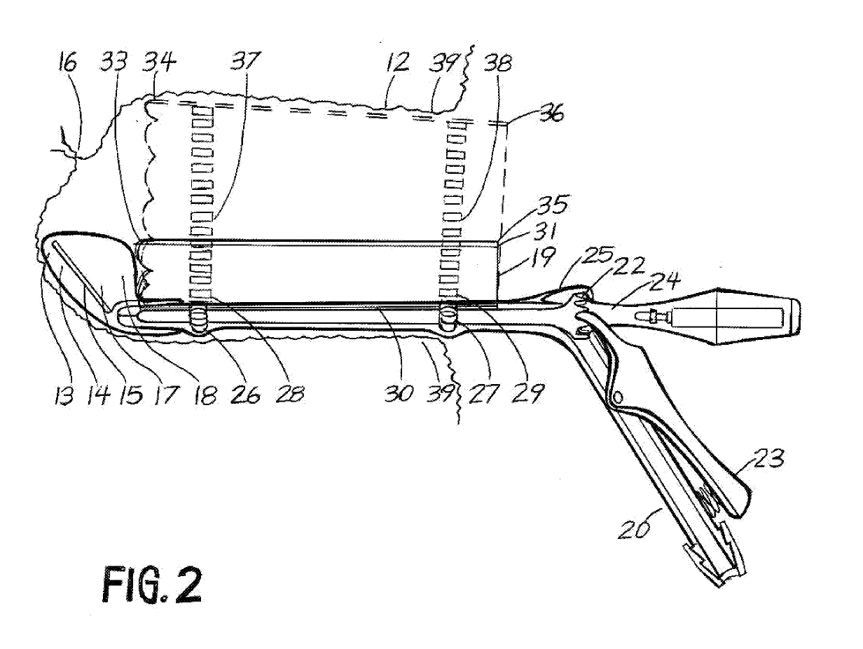

FIG. 2 shows a side view of an embodiment of the present invention tunnel vision gynecological examination device in a rolled configuration (solid lines) and an expanded configuration (dotted lines) illustrating the 10 tunnel vision speculum inserted into the 11 vaginal opening with the 13 rounded tip of the 14 orifice opener positioned deep inside the 12 vaginal lining where a 15 mirror mounted to the inside of the 14 orifice opener is located in a perfect position and angle below the 16 cervix to provide a 17 reflection of cervix to the gynecologist viewing through the 18 spreader flange and the 19 spiral rolled clear tunnel displaying a complete 360 degree view of the 12 vaginal lining and the 16 cervix and the 17 reflection of the cervix at once. The gynecologist controls the movement of the 10 tunnel vision speculum by grasping the 20 handle. In order to gain a wider view of the 12 vaginal lining, the gynecologist rotates the finger knob, after disengaging the 22 ratchet teeth by depressing the 23 ratchet release on the 20 handle, which causes the rotation of the 24 gear drive shaft within the 25 drive shaft sleeve causing the 26 front drive gear teeth and the 27 rear drive gear teeth to engage and move the 28 front drive gear holes and the 29 rear drive gear holes within the 30 alignment guide slot causing the 31 rolled tunnel to move in a manner that increases the diameter of the spiral resulting in an 32 unrolled tunnel where the 33 rolled forward diameter is enlarged to form the 34 unrolled forward diameter and the 35 rolled rear diameter is enlarged to form the 36 unrolled rear diameter causing the tunnel to expand in the 12 vaginal lining to give the gynecologist an excellent view of the vaginal orifice for examinations, treatments, operations and tests. The tunnel vision speculum is provided with a 37 forward minimum diameter locking hole and a 38 rear minimum diameter locking hole in order to limit the minimum diameter the 19 spiral rolled clear tunnel may be reduced in size to avoid taking the spiral wound plastic past the point where the elastic memory of the plastic flex capability is exceeded and the plastic will no longer spring out into a tunnel shape again. The 34 unrolled forward diameter is larger than the 36 unrolled rear diameter causing a conical shape to be formed inside the 12 vaginal lining where the 39 vaginal pinching muscle has a tendency to hold the device inside the 11 vaginal opening until the 32 unrolled tunnel is reduced in size to form the 31 rolled tunnel which is small enough in diameter to be removed.

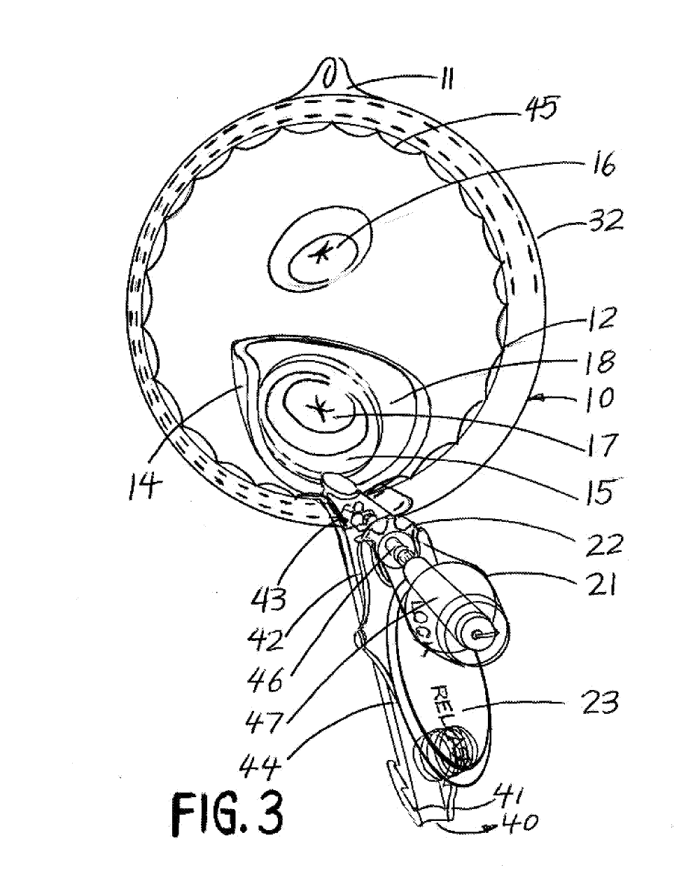

FIG. 3 shows an end view of an embodiment of the present invention tunnel vision gynecological examination device as seen from the viewpoint of a gynecologist doing a vaginal examination illustrating the 10 tunnel vision speculum inserted into the 11 vaginal opening looking into the end of the 32 unrolled clear tunnel formed inside the 12 vaginal lining. The view down the tunnel reveals the 360 degree display of the color and texture of the vaginal tissue surrounding the tunnel and at the end of the tunnel is a view of the 17 reflection of the cervix in the 15 mirror located within the 18 spreader flange of the 14 orifice opener. When the 23 ratchet release on the 20 handle is squeezed, the 22 ratchet teeth become free to rotate and the 21 finger knob can be rotated to enlarge the small diameter 31 rolled tunnel to the larger 32 unrolled tunnel. This enlargement allows the visual assessment of the condition of the front of the 16 cervix using direct eye contact and the rear of the 16 cervix using the 17 reflection of the cervix in the 15 mirror. The 10 tunnel vision speculum may be provided with a 40 vacuum source in order to remove fluids through a 41 vacuum tube that is connected to a 42 vacuum manifold drain that suctions fluids that collect in 43 vacuum manifold intake slots in the 44 vacuum manifold. The 10 tunnel vision speculum is provided with a flexible 45 soft rounded conformal edge to safely and comfortably expand within the 12 vaginal lining to afford the greatest access with the minimum discomfort. The 10 tunnel vision speculum may be equipped with a 46 light source and 47 light source power connector for the purpose of illuminating the interior of the 12 vaginal lining for improved ocular acuity.

The preferred embodiments herein disclosed are not intended to be exhaustive or to unnecessarily limit the scope of the invention. The preferred embodiments were chosen and described in order to explain the principles of the present invention so that others skilled in the art may practice the invention. Having shown and described preferred embodiments of the present invention, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

What is claimed is:

1. A gynecological examination device and method for the opening of the vagina for viewing of the vaginal lining and examination and treatment of the cervix which opens the vagina by inserting a rolled sheet of flexible material which is unrolled inside the vagina forming an expanding clear tunnel, which enlarges to display a 360 degree tunnel view of the vaginal lining and displays the cervix at the end of the tunnel.

2. The gynecological examination device of claim 1 with one or a plurality of the following features or functions; a mirror to offer an additional angle of view of the cervix for improved gynecological diagnosis and treatment, lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

3. An orifice examination device and method for opening of any orifice for viewing of the orifice lining and examination and treatment of the interior of the orifice which opens the orifice by inserting a rolled sheet of flexible material which is unrolled inside the orifice forming an expanding clear tunnel, which enlarges to display a 360 degree tunnel view of the orifice lining and displays the interior of the orifice at the end of the tunnel.

4. The orifice examination device of claim 3 with one or a plurality of the following features or functions; a mirror to offer an additional angle of view of the orifice for improved diagnosis and treatment, lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

5. An orifice examination device placed inside the orifice that transforms from an easy to insert shape to an enlarged hollow clear tunnel that allows the display of a 360 degree tunnel view of the orifice lining and displays the interior of the orifice at the end of the tunnel.

6. The orifice examination device of claim 5 with one or a plurality of the following features or functions; a mirror to offer an additional angle of view of the orifice for improved diagnosis and treatment, lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

7. An orifice examination device of claim 3 adapted to all mammal's orifices, incisions or holes.

8. An orifice examination device of claim 3 adapted to open an incision and become a retractor to hold tissue open during an operation.

9. An orifice examination device of claim 3 adapted to all animal's orifices, incisions or holes.

10. An orifice examination device of claim 3 adapted to all animal's, plant's and mineral's orifices, incisions or holes.

11. An orifice examination device of claim 3 adapted as a device that can be scaled in size to open the hole from a bullet wound to expose the bullet's location and provide a clear, light illuminated, open tunnel to facilitate removal of the bullet, while a local coagulant coating on the outside surface of the tunnel stops the bleeding of the bullet wound into the hole, while a local anesthetic reduces the pain of the wound.
